(19) United States
(12) Patent Application Publication
(10) Pub. No.: US 2025/0064347 A1
(43) Pub. Date: Feb. 27, 2025

(54) DETECTION OF MEDICINES AND METABOLITES IN URINE

(71) Applicant: Zemplee Inc., Campbell, CA (US)

(72) Inventors: Aparna Pujar Anthapur, Campbell, CA (US); Samyak Jain, Campbell, CA (US)

(73) Assignee: Zemplee Inc., Campbell, CA (US)

(21) Appl. No.: 18/726,360

(22) PCT Filed: Jan. 3, 2023
(86) PCT No.: PCT/US2023/060030

§ 371 Date: Jul. 2, 2024

Related U.S. Application Data
(60) Provisional application No. 63/296,105, filed on Jan. 3, 2022.

(51) Int. Cl. A61B 5/145; A61B 5/00; A61B 5/1171; A61B 5/1172; A61B 5/1477; A61B 10/00

(52) U.S. Cl. CPC A61B 5/14507; A61B 5/0077; A61B 5/1172; A61B 5/1176; A61B 5/1477; A61B 5/4833; A61B 5/6844; A61B 5/6891; A61B 10/00; A61B 10/007; A61B 2010/0006

(57) ABSTRACT

Various examples include an apparatus and a method to detect various medicines and their metabolites from a urine stream of a patient. In one example, an apparatus that can be attached to a toilet to detect various medicines and their metabolites from a urine stream of a patient includes a device carrier having at least one reagent-infused strip, an absorbent pad, and a transfer tube to direct at least portions of the urine stream from the absorbent pad to the at least one reagent-infused strip. A camera is used to collect an image from each of the at least one reagent-infused strips and produce information therefrom. A signaling device comprising a combination transponder and signaler is configured to transmit information received from the camera to a storage device for later analysis of the various medicines and their metabolites. Other apparatuses, devices, and methods are disclosed.

### CLAIM OF PRIORITY

This patent application claims priority to U.S. Provisional Application Ser. No. 63/296,105, entitled, “DETECTION OF MEDICINES AND METABOLITES IN URINE,” filed 3 Jan. 2022, the disclosure which is incorporated herein by reference in its entirety.

### TECHNOLOGY FIELD

The disclosed subject matter is related generally to the field of detecting various types of medicines and metabolites in urine samples of a human patient. More specifically, in various embodiments, the disclosed subject matter discloses a device that can be attached to a toilet to detect various medicines and their metabolites from the urine stream of the patient.

### BACKGROUND

Medication Compliance is a critical part of the efficacy of aspects of medical care. Only by determining whether medication is being consumed can care providers accurately provide further medical care. For example, poor compliance among the elderly is a public-health concern as it greatly increases the cost of healthcare, increases medicine wastage, and results in a significant worsening of diseases with a higher chance of disability or death. As the geriatric population grows, there is an increasing call to improve healthcare delivery systems, particularly for chronic diseases. Chronic diseases, or even multiple diseases, can require a complex medication-regime. A number of factors, such as a higher cost of therapy, forgetfulness, lack of familial/social support or care, and/or side-effects can all lead to reduced medication compliance.

Despite the urgency and critical nature of the issue of identification of medication compliance, there is currently no good solution currently available to determine whether compliance actually occurs or is complete. Instead, the vast majority of care providers depend nearly exclusively on self-reporting from patients to ensure medication compliance. The self-reporting of compliance from patients is neither accurate nor reliable.

The method of detection of medication is desirably quick, reliable, and compact. In prior art approaches, laser spectroscopy systems have been considered. However, such laser-based systems are inadequate due, at least partially, to the difficulty of data collection. Urine streams generally flow too quickly, have too many solute particles of too many varying sizes, and do not follow a set path such that the laser-based systems could be effective. Lasers also have an associated electricity cost to operate, and this cost would be borne entirely by the consumer, which is not ideal. Spectroscopic analysis equipment also requires clean environments and/or frequent cleaning, neither of which is suitable for a home-use product.

Consequently, an unobtrusive, passive, and non-invasive method of medication compliance is desirable. Such a method, and a related mechanism by which the method can be accomplished, reduces or eliminates the need for self-reporting. Moreover, such a method should not be overly invasive or provide little to no discomfort for the patient.

The background description provided here is for the purpose of generally presenting the context of the disclosed subject matter. Work of the presently named inventors, to the extent that it is described in this background section, as well as aspects of the description that may not otherwise qualify as prior art at the time of filing, are neither expressly nor impliedly admitted as prior art against the present disclosure. Consequently, the information described in this section is provided to offer the skilled artisan a context for the following disclosed subject matter and should not be considered as admitted prior art. The information described in this section is therefore provided to offer the skilled artisan a context for the following disclosed subject matter and should not be considered as admitted prior art.

### SUMMARY

In various embodiments, an apparatus and a method to detect various medicines and their metabolites from a urine stream of a patient are disclosed. In one example, an apparatus that can be attached to a toilet to detect various medicines and their metabolites from a urine stream of a patient includes a device carrier having at least one reagent-infused strip, an absorbent pad, and a transfer tube to direct at least portions of the urine stream from the absorbent pad to the at least one reagent-infused strip. A camera is used to collect an image from each of the at least one reagent-infused strips and produce information therefrom. A signaling device comprising a combination transponder and signaler is configured to transmit information received from the camera to a storage device for later analysis of the various medicines and their metabolites.

In various embodiments, the disclosed subject matter includes an apparatus that can be attached to a toilet to detect various medicines and their metabolites from a urine stream of a patient. The apparatus includes a device carrier having at least one reagent-infused strip, an absorbent pad, and a transfer tube to direct at least portions of the urine stream from the absorbent pad to the at least one reagent-infused strip. The apparatus further includes a camera to collect an image from each of the at least one reagent-infused strips and produce information therefrom, and a signaling device comprising a combination transponder and signaler to transmit information received from the camera to a storage device for later analysis of the various medicines and their metabolites.

In various embodiments, an exemplary method for detecting various medicines and their metabolites from a urine stream of a patient is disclosed. The exemplary method includes obtaining a biometric identification of the patient and making a determination as to whether the patient is biologically male or biologically female. Based on a determination the patient is biologically male, making a further determination whether the biologically-male patient will be urinating from a seated position or a standing position. A device carrier having at least one reagent-infused strip, an absorbent pad, and a transfer tube is moved to a proper position based on the determination of a seated patient and a standing patient. A determination is made that a urination process is completed. A camera is used to collect an image from each of the at least one reagent-infused strips and information therefrom is produced. The information received from the camera is transmitted to a storage device for later analysis of the various medicines and their metabolites.

In various embodiments, a method for detecting various medicines and their metabolites from a urine stream of a patient is disclosed. The method including obtaining a biometric identification of the patient, and making a determination as to whether the patient is biologically male or biologically female. Based on a determination the patient is biologically male, making a further determination whether the biologically-male patient will be urinating from a seated position or a standing position. The method further includes moving a device carrier having at least one reagent-infused strip, an absorbent pad, and a transfer tube to a proper position to capture at least a portion of the urine stream based on the determination between a seated patient and a standing patient. A determination is made as to whether a urination process is completed. A camera is used to collect an image from each of the at least one reagent-infused strips and producing information therefrom.

In various embodiments, the disclosed subject matter includes a sample-collection device to detect various medicines and their metabolites from a urine stream of a patient. The device includes a device carrier having at least one reagent-infused strip to collect at least a portion of the urine stream, and a camera to collect an image from each of the at least one reagent-infused strips and produce information therefrom.

### BRIEF DESCRIPTION OF FIGURES

FIG. 1 shows an example of a sample-collection device, in accordance with various embodiments of the disclosed subject matter;

FIG. 2A shows an example of a deployment mechanism that can be used with the sample-collection device of FIG. 1, in accordance with various embodiments of the disclosed subject matter;

FIG. 2B shows an example of the sample-collection device of FIG. 1 and the deployment mechanism of FIG. 2A used in situ in an exemplary deployed-position, in accordance with embodiments of the disclosed subject matter;

FIGS. 3A and 3B show examples of biometric-identification devices to identify a user of the sample-collection device of FIG. 1, in accordance with various embodiments of the disclosed subject matter;

FIG. 4A shows an example of a urination-detection device, in accordance with various embodiments of the disclosed subject matter;

FIG. 4B shows an alternative location of a deployed position of the sample-collection device of FIG. 1, in accordance with various embodiments of the disclosed subject matter;

FIG. 4C shows a retracted position of the sample-collection device of FIG. 1, in accordance with various embodiments of the disclosed subject matter; and

FIG. 4D shows an alternative position of a camera used to view the sample-collection device of FIG. 1, in accordance with various embodiments of the disclosed subject matter.

FIGS. 5A and 5B show an example of a method to detect various medicines and their metabolites from a urine stream of a patient.

### DETAILED DESCRIPTION

The description that follows includes illustrative examples, devices, and apparatuses that embody various aspects of the disclosed subject matter. In the following description, for purposes of explanation, numerous specific details are set forth in order to provide an understanding of various embodiments of the inventive subject matter. It will be evident however, to those of ordinary skill in the art, that various embodiments of the disclosed subject matter may be practiced without these specific details. Further, well-known structures, materials, and techniques have not been shown in detail, so as not to obscure the various illustrated embodiments. As used herein, the terms “about” or “approximately” may refer to values that are, for example, within ±10% of a given value or range of values.

In various embodiments, an unobtrusive, passive, and non-invasive method of medication compliance, and a related device by which the method can be accomplished, is disclosed. Therefore, at least one goal of the disclosed subject matter is to create a device or apparatus to detect medication in the urine, either automatically or semi-automatically, to obtain proof-of-patient-adherence with medication objectives.

In general, chemical-spot-tests using paper or a pad pre-impregnated with a correct reagents presents a basis for the disclosed subject matter. Such chemical-spot-tests are quick, inexpensive, and are simple to analyze. Chemical-spot-tests test for a specific solute, and thus are specific tests. Such tests require only prior knowledge of the solutes and which chemicals will react with them (some of which are discussed below in more detail). Thus, in simple terms. a simple dipstick; a strip of paper attached to a stick, loaded with the correct reagent or reagents, and placed in the path of the urine stream can be used. This simple concept forms the basic structure of the device disclosed herein.

In various embodiments, a device attached to a toilet that detects various medicines and their metabolites in the urine stream of the patient is disclosed. In one embodiment, the urine is absorbed by an absorbent pad and then wicked up (e.g., by capillary action) onto testing pads (e.g., sponge-like pads or papers) that are each pre-impregnated with one or more reagents that respond to specific medication and their metabolites. Upon reaction, each pad undergoes a visual change, detected as an image by a camera that relays the information to a server (either local or remote). The image, or data derived from the image, is logged and forwarded to appropriate care-providers. The testing pads are then discarded into the toilet. A new substrate carrying one or more testing pads is then loaded into the device automatically.

Therefore, the process works by first identifying reactions which the medication and its metabolites can undergo to give visual changes when in the presence of the medications as carried in from the urine stream. In embodiments, the reagents for each reaction may be loaded side-by-side on to a testing strip. When the testing strip interacts with urine, depending on whether or not the patient has consumed the medication and contains those metabolites in the urine stream of the patient, the appropriate reagents on the test strip react and change color, which can be observed and imaged by the camera. Thus, medication compliance may be determined without the patient even noticing.

As is known in the relevant art, medicines may contain both active ingredients and passive ingredients. Upon ingestion, the active ingredients are absorbed into the body and metabolized either in the liver, the plasma, or the intestines by action of Cytochrome P450 (CYP, CYP450, or CYP₄₅₀) enzymes. For the purpose of illustration herein, Cytochrome P450 2E1 (CYP2E1) is a member of the cytochrome P450 mixed-function oxidase system, which is involved in the metabolism of xenobiotics in the body. The enzyme CYP2E1 is involved in Paracetemol (also known as acetaminophen) metabolism.

The metabolized products (“metabolites”) are the chemicals that are detected in the urine, along with the unchanged drug if possible. Thus, the first step is to properly identify the metabolites, the method of excretion, and then consider whether or not they can be detected.

### Examples of Medication Analysis

Examples of medicines considered herein are the most common ones—Acetaminophen, Aspirin, Metformin, Ibuprofen, Glipizide, Metoprolol, Enalapril, Atorvastatin, Fluvastatin, Lovastatin, Pitavastatin, Pravastatin, Rosuvastatin, and Simvastatin are discussed below.

Aspirin: Aspirin is metabolized in the plasma and the liver. The main metabolic products are salicyluric acid, ether or phenolic glucuronide, and ester or acyl glucuronide. Aspirin is excreted through the kidneys in the form of free salicylic acid, salicyluric acid, as well as phenol or acyl glucuronides. Aspirin can be detected using a Trinder reagent, which turns red upon contact.

Ibuprofen: Ibuprofen is metabolized in the liver. The main metabolic products are 2-hydroxy-ibuprofen (2-OH-IBU) and ibuprofen carboxylic acid (CBX-IBU). Ibuprofen is excreted through the kidneys in the form of its metabolites or their conjugates. There is no known chemical test for its metabolites.

Metformin: Metformin is not known to metabolize in the body and is absorbed and excreted in its unchanged form through the kidneys. Metformin can be detected using a solution of Nickel (II) Chloride, which results in the formation of an orange precipitate.

Enalapril: Enalapril is metabolized in the liver and forms Enalaprilat. Both unchanged Enalapril and Enalaprilat are excreted through the kidneys and are found in both urine and feces. Enalapril can be detected by using a combined solution of Potassium Iodate and Potassium Iodide, which will turn yellow.

Atorvastatin: Atorvastatin is metabolized by CYP450 in the liver and intestines. The metabolic products of Atorvastatin are ortho-and para-hydroxylated derivates. Atorvastatin is eliminated via biliary excretion with less than 1% being eliminated via the urine. The extremely low concentration of Atorvastatin and its metabolites in urine makes detection via chemical test difficult.

Simvastatin: Simvastatin is metabolized by CYP450 in the liver and intestines. The metabolic products of Simvastatin are B-hydroxy acid metabolite and its 6′-hydroxy, 6′-hydroxymethyl, and 6′-exomethylene derivatives. Simvastatin is excreted through the kidneys and is found in large amounts in both urine and feces. However, there is no known chemical test for Simvastatin or its metabolites.

Metoprolol: Metoprolol is metabolized through action of CYP and forms the metabolites alpha-hydroxy metoprolol, o-demethyl metoprolol, and deisopropyl metoprolol. Metoprolol is excreted through the kidneys and is found in urine. However, there is no known chemical test for Metoprolol or its metabolites.

Glipizide: Glipizide undergoes hepatic metabolism in the liver. The major metabolites of Glipizide are 3-cis-hydroxyglipizide, 4-trans-hydroxyglipizide, and 4-trans-OH-glipizide. Glipizide is excreted through the kidneys and eliminated via the urine and feces. There are currently no known chemical tests for sulfonylurea compounds (which comprises Glipizide) that can be utilized.

Therefore, the medicines which can theoretically be detected are Aspirin, Metformin, and Enalapril. However, another factor to consider is that medication contains passive/inert ingredients, which are also excreted and can potentially be detected. This is not feasible in practice because many medicines contain common inert ingredients so testing for them does not identify which medicine was consumed and beats the point of medication compliance. Additionally, these inert ingredients are also found in other common foodstuffs. For example, stearic acid is found in both Glipizide and chocolate, Butylated hydroxyanisole is found in both Simvastatin and butter. So the only way to ensure medication compliance by testing for these ingredients is by having perfect control of the patients diet, which is not ideal or even possible. Thus, we can only rely on the active ingredients in each medicine as these are the only reliable indicators of specificity. However, a prior recording of the medication schedule and list of medications prescribed can be used to determine the

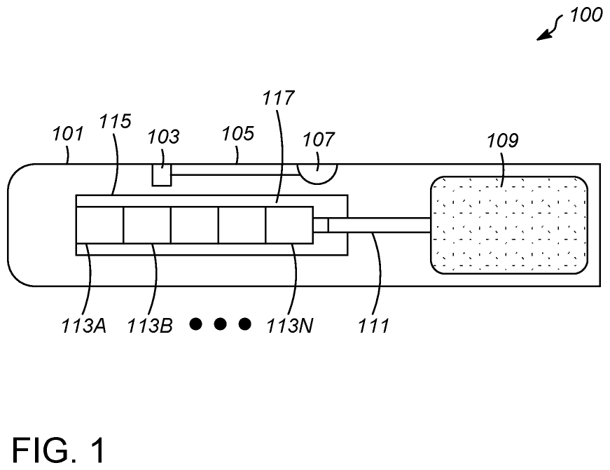

With reference now to FIG. 1, an example of a sample-collection device 100 in accordance with various embodiments of the disclosed subject matter is shown. The sample-collection device 100 is shown to include a device carrier 101, a signaling device 103, a communications interconnect 105, a camera 107, and an absorbent pad 109. The sample-collection device 100 is also shown to include a number of reagent-infused strips 113A, 113B, . . . , 113N. The reagent-infused strips 113A, 113B, . . . , 113N are mounted on a strip substrate 117 and are visible, in this embodiment, through an opening 115 in an upper portion if the device carrier 101. A tube 111 (e.g., a thin capillary tube or transfer tube comprising various types of plastic, glass, or other non-contaminating materials) is hydraulically-coupled to conduct fluids from the absorbent pad 109 to the reagent-infused strips 113A, 113B, . . . , 113N. The tube 111 serves to wet each of the reagent-infused strips 113A, 113B, . . . , 113N with urine, thereby causing reactions in the associated reagent if the correct solutes are present. In an alternative embodiment, the strip substrate 117 can be positioned such that the urine hits the reagent-infused strips 113A, 113B, . . . , 113N directly. In this alternative embodiment, the absorbent pad 109 and the tube 111 may be considered to be optional elements.

In various embodiments, the device carrier 101 comprises, for example, one or more hard plastic materials. The one or more hard plastic materials may be machined to carry the other components of the sample-collection device 100 or may be formed or otherwise produced (e.g., such as a thermosetting plastic or thermosetting polymer). However, in other embodiments, the device carrier 101 may comprise various types of metallic materials, such as a stainless-steel alloy (e.g., a 304 or 316L grade stainless steel). In general, the device carrier 101 may be selected to comprise a material that prevents any external environmental contamination and is not affected by urine or an environment in which the device carrier 101 is located.

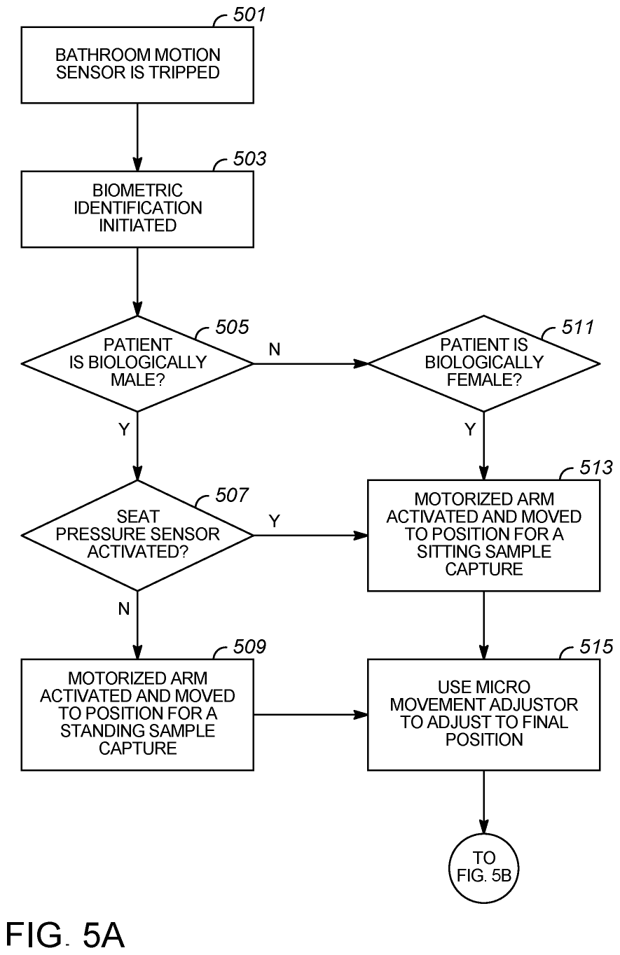

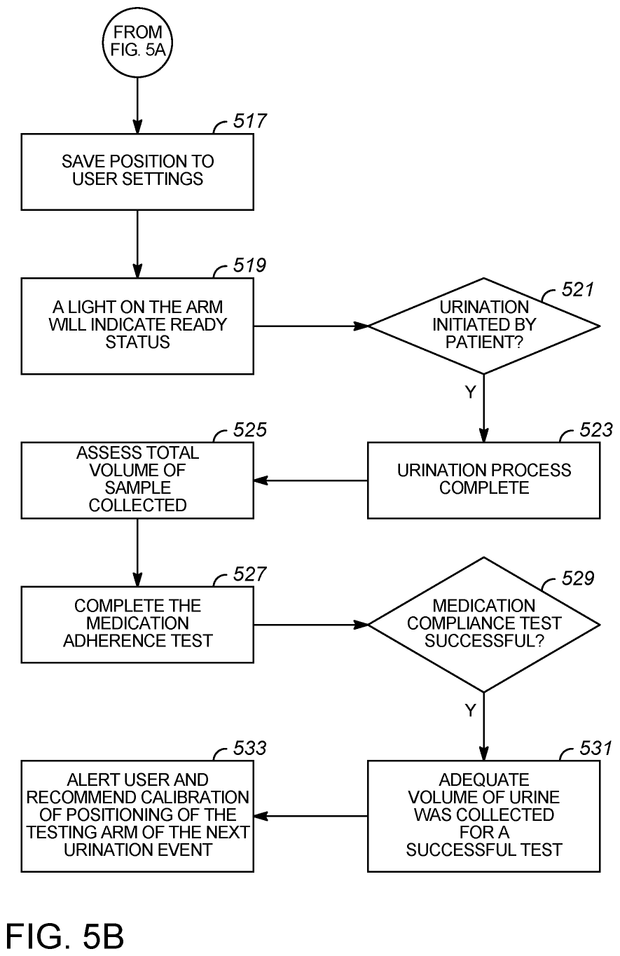

The absorbent pad 109 is positioned (e.g., as described below) to intercept at least a portion of urine flow. As discussed in more detail below with reference to FIGS. 5A and 5B, there are at least three possibilities to be considered for positioning the absorbent pad 109: a standing biological-male, a seated biological-male, and a seated biological-female. The standing biologically-male can be trained to guide the urine stream towards the absorbent pad 109 so as to ensure adequate absorption. However, in the seated positions, the motorized arm will be guided into position based on an identification of the patient. A control mechanism can be used to make smaller adjustments to the position. A remote control device (not shown but understandable to a person of ordinary skill in the art upon reading and understanding the disclosed subject matter) can be used to operate the arm and can provide a function to save the position settings to a local data storage device (e.g., an internal flash-memory or other non-volatile memory device) for an individual patient. A pressure sensor on the substrate of the arm or a toilet-seat ring (described in more detail below) can be used to detect occupancy of the seat and further distinguish the standing position from the seated position and register the correct position.

The tube 111 leads from the absorbent pad 109 to the reagent-infused strips 113A, 113B, . . . , 113N. Therefore, each of the reagent-infused strips 113A, 113B, . . . , 113N absorbs at least a portion of the intercepted urine flow. For example, in one specific exemplary embodiment, the absorbent pad 109 may comprise a simple sponge or sponge-like material that is positioned to absorb urine as the patient is urinating. The urine then flows from the absorbent pad 109 toward the reagent-infused strips 113A, 113B, . . . , 113N through the capillary tube by capillary action. The absorbent pad 109 may further act as a filter, helping to remove or reduce the number of any proteins or bacteria in the urine that may affect performance of the assay, leaving only the urine (mostly water) and smaller metabolites.

The strip substrate 117 may comprise a number of materials that are suitable for mounting the reagent-infused strips 113A, 113B, . . . , 113N such as various types of plastic. In one specific exemplary embodiment, the strip substrate 117 may comprise a lightweight cardboard or thin plastic material. Since the reagent-infused strips 113A, 113B, . . . , 113N and the strip substrate 117 are discarded after each use (e.g., into a toilet on which the sample-collection device 100 is mounted as described below), the reagent-infused strips 113A, 113B, . . . , 113N and the strip substrate 117 may be formed from inexpensive materials.

The reagent-infused strips 113A, 113B, . . . , 113N may comprise, for example, nonwoven fibers that are pressed into, for example, indentations in the strip substrate 117 or otherwise adhered (e.g., by an adhesive or other binding agent) onto the strip substrate 117. Each of the reagent-infused strips 113A, 113B, . . . , 113N is coated or infused with a different reagent for each medicine or metabolite to be detected, as described herein.

Although FIG. 1 indicates five separate reagent-infused strips, a larger or smaller number of strips may be used depending on a number of medicines and their metabolites to be detected. For example, in one specific exemplary embodiment, only one reagent-infused strip may be present in order to detect only a single medicine (e.g., metformin, such as Glucophage®). In other specific exemplary embodiments, more than five separate reagent-infused strips may be used. Therefore, the strip substrate 117 may contain one or more separate reagent-infused strips.

In general, the reagents coated or infused into the separate reagent-infused strips interact with the medicines and their metabolites in a series of steps, ultimately resulting in a color change of the reagent-infused or coated strip. An example embodiment is pH paper that is modified (e.g., pre-impregnated) with the correct reagents to test for medicines, as opposed to a pH level. Further, although not shown but understandable to a person of ordinary skill in the art upon reading and understanding the disclosed subject matter, a desiccant packet may be placed proximate to the reagent-infused strips 113A, 113B, . . . , 113N to maintain a relatively moisture-free environment to assist in the testing process. Therefore, the desiccant packet helps to keep portions (e.g., an interior) of the sample-collection device 100 free from excess moisture.

Although the strip substrate 117 containing the reagent-infused strips 113A, 113B, . . . , 113N is shown through the opening 115 in an upper portion if the device carrier 101, such an arrangement is shown merely as one possible arrangement. In other embodiments, the opening 115 may be arranged on a lower portion (e.g., not visible to an observer looking down toward the sample-collection device 100) of the device carrier 101. As discussed below, the reagent-infused strips 113A, 113B, . . . , 113N only need to be visible to the camera 107, and not necessarily all at once. That is, each of the reagent-infused strips 113A, 113B, . . . , 113N only need to be viewable by the camera 107 at some point in time (e.g., as the strip substrate 117 containing the reagent-infused strips 113A, 113B, . . . , 113N is being disposed of after use into the toilet).

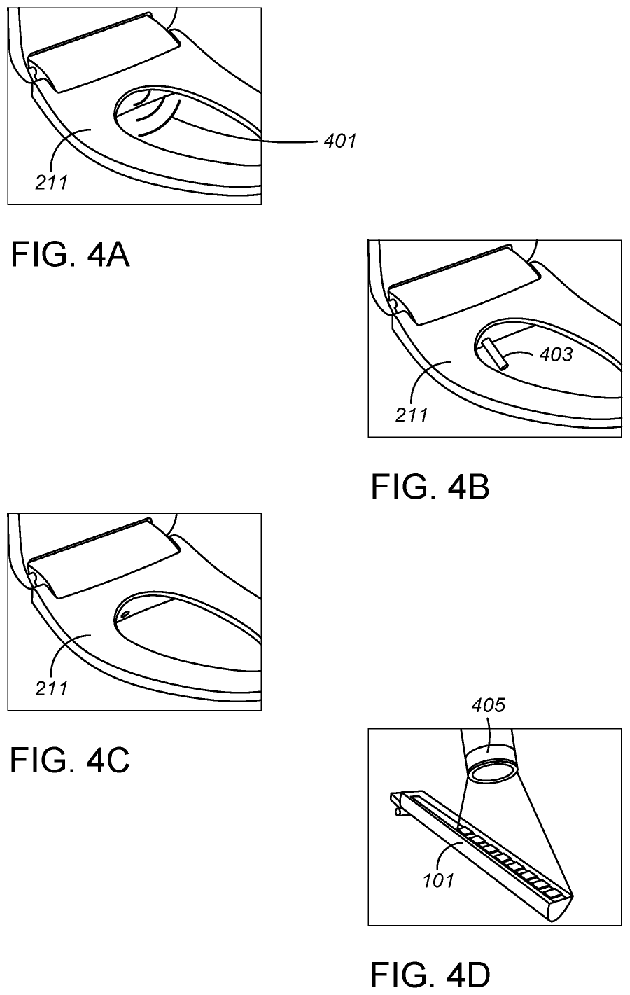

For example, the camera 107 may collect an image from each of the reagent-infused strips 113A, 113B, . . . , 113N as they pass by the camera 107 as the strip substrate 117 containing the reagent-infused strips 113A, 113B, . . . , 113N is disposed of after use. In other embodiments, the camera 107 may be positioned to move in a longitudinal direction (e.g., by a linear actuator or related device) along the device carrier 101 to view each of the reagent-infused strips 113A, 113B, . . . , 113N individually. In other embodiments, the camera 107 may be fitted with a wide-angle lens such that the camera 107 may view and collect data from each of the reagent-infused strips 113A, 113B, . . . , 113N in a single shot. In still other embodiments, a camera 405 may be positioned distal to the device carrier 101 (e.g., as shown in fog 4D and discussed below). In the distal position shown in FIG. 4D as an example, the camera 405 may view and collect data from each of the reagent-infused strips 113A, 113B, . . . , 113N in a single shot or in a series of shots. Therefore, in various embodiments, the opening 115 is open such that the reagent-infused strips 113A, 113B, . . . , 113N are viewable by the camera 107. However, the opening 115 may not be visible external to the sample-collection device 100.

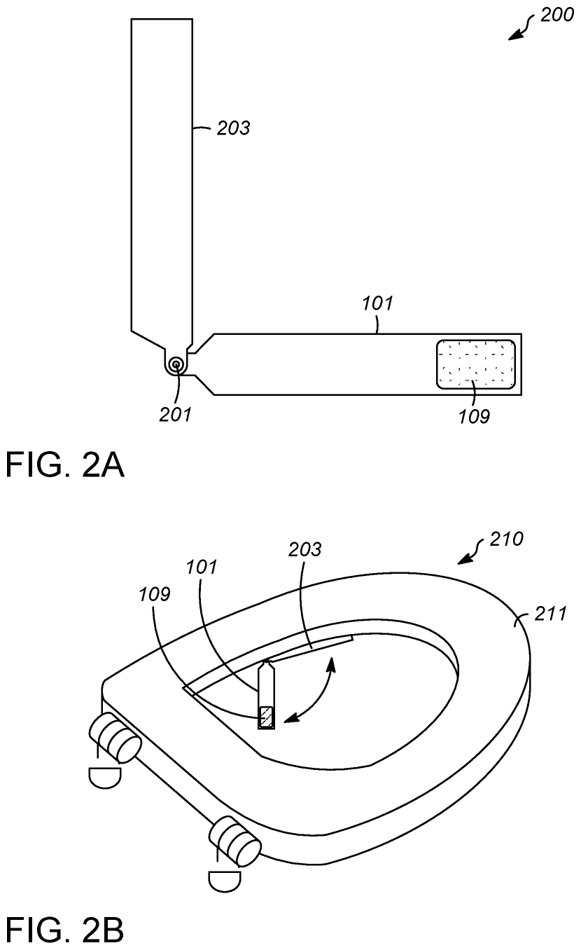

Although not shown explicitly, upon reading and understanding the disclosed subject matter, a person of ordinary skill in the art will recognize a number of means for dispensing and disposing of the strip substrate 117 after use. For example, in one embodiment, a small capstan and pinch roller device can be embedded along one edge and within the device carrier 101 to dispose of used substrates and reload new ones. New ones of the strip substrate 117 may be loaded from, for example, a storage supply located on the underside of a toilet ring on which the sample-collection device 100 is mounted (e.g., as described below with reference to FIG. 2B). In other embodiments, a roll of the strip substrates 117 may supply separable ones of the substrates. In other embodiments, a linear actuator or related device may advance used versions of the strip substrate 117 out of the device carrier 101 and, subsequently, load a new strip substrate 117.

The camera 107 views changes in color of each of the reagent-infused strips 113A, 113B, . . . , 113N after exposure to the urine. In various embodiments, the camera 107 may comprise a small, wireless camera (e.g., a button camera). A resolution level of the camera may range from, for example, 640×480 pixels (e.g., approximately 307,000 pixels or less) to 3840×2160 (e.g., approximately 8.3 million pixels, a “4K” camera) or more. Considerations for the camera 107 primarily include a color camera, so as to view color changes on the reagent-infused strips 113A, 113B, . . . , 113N, and a compact size so as to fit in or proximate to the sample-collection device 100 (e.g., FIG. 4D, discussed below, shows an embodiment of a camera mounted proximate to, but separate from, the sample-collection device 100).

With continuing reference to FIG. 1, in various embodiments the camera 107 may be coupled electrically to the signaling device 103 via the communications interconnect 105. In various embodiments, the communications interconnect 105 may comprise an electrical ribbon connection or antenna for wireless communications. One or more images captured by the camera are, for example, transmitted and/or stored and logged. The captured images allow a medical-care provider to construct a medication profile for the patient.

Therefore, the signaling device 103 may comprise a combination transponder and signaler to transmit information (e.g., wirelessly) received from the camera to a remote storage device or uploaded to, for example, a cloud-based server. Either the remote storage device and/or the cloud-based server may be located at a facility of a medical-service provider. In other embodiments, the signaling device 103 may comprise, for example, a Bluetooth®-type of telecommunications device. Such types of transponder/signaler devices and Bluetooth®-types of telecommunications devices are known independently in the telecommunications arts.

FIG. 2A shows an example of a deployment mechanism 200 that can be used with the sample-collection device 100 of FIG. 1, in accordance with various embodiments of the disclosed subject matter. The deployment mechanism 200 is shown to include a bracket 203, which may be mountable to, for example, an underside of a rim of a toilet seat ring. As discussed above, the bracket 203 may be configured (e.g., physically sized) to store a supply of strip substrates 117. A storage container may also be located separately in other positions under a toilet ring 211 (see FIG. 2B).

A driving component 201 allows the device carrier 101 of the sample-collection device 100 to be positioned into a urinal stream, as discussed in more detail with reference to FIG. 2B, below. The driving component 201 is therefore capable of extending and retracting the device carrier 101, and at predetermined angles for a given patient. In various embodiments, the driving component 201 may comprise a rotatable motor. In a specific exemplary embodiment, the driving component comprises an Arduino stepper motor. Other mechanisms for deploying the device carrier 101 into a urine stream are described with reference to FIG. 4B, below.

FIG. 2B shows an example of the sample-collection device 100 of FIG. 1 and the deployment mechanism 200 of FIG. 2A used in situ 210 in an exemplary deployed-position, in accordance with embodiments of the disclosed subject matter. The device carrier 101 is shown in FIG. 2B with the bracket 203 mounted to an underside of the toilet ring 211. An angle that the device carrier 101 swings away from the toilet ring 211 can be determined based on factors such as the sex and position (e.g., standing versus seated) of the user.

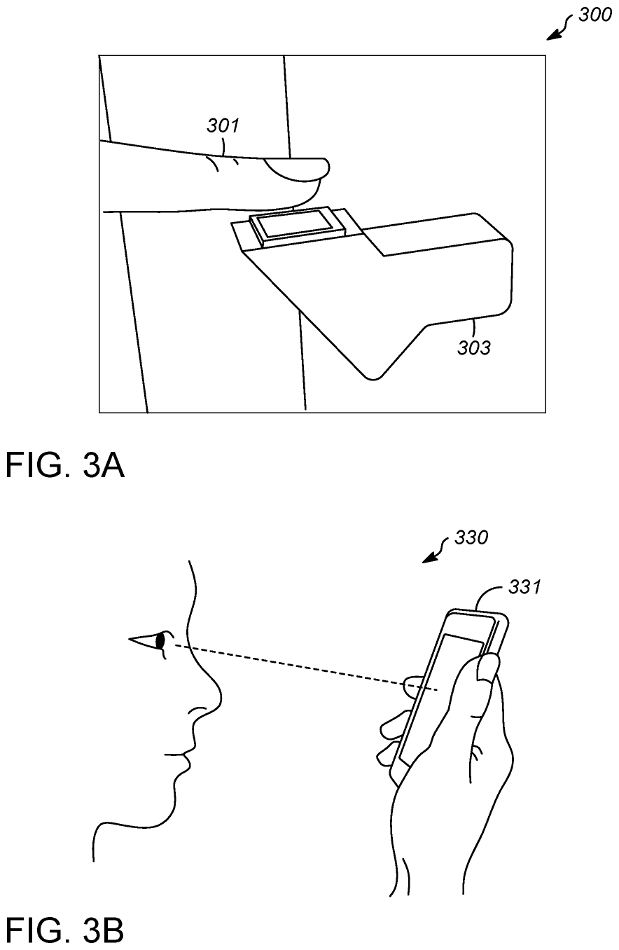

FIGS. 3A and 3B show examples of biometric-identification devices 300, 330 to identify a user of the sample-collection device 100 of FIG. 1, in accordance with various embodiments of the disclosed subject matter. For example, FIG. 3A shows a fingerprint scanner 303 that determines the user based on a scan of a finger 301 of the user. In embodiments, the fingerprint scanner 303 may form all of or a portion of a flush lever of the toilet onto which the sample-collection device 100 is mounted.

FIG. 3B shows a facial or retinal scanner type of biometric-identification device. In this embodiment, the user is looking at an application that may be downloaded and installed on an identification device 331 or other type of device capable of running a biometric-identification application, such as a smartphone or tablet. In other embodiments, the identification device 331 may be mounted on or be formed integral with the toilet on which the sample-collection device 100 is installed.

FIG. 4A shows an example of a urination-detection device 401, in accordance with various embodiments of the disclosed subject matter. In this example, an infrared or ultrasonic scan can be used to detect the presence of a urine flow after the user has been identified by, for example, one of the biometric-identification devices 300, 330 of FIG. 3A or 3B. Based upon reading and understanding the disclosed subject matter, a person of ordinary skill in the art will recognize a number of other urine flow detection devices that may be used with the disclosed subject matter as well.

FIG. 4B shows an alternative location 403 of a deployed position of the sample-collection device 100 of FIG. 1, in accordance with various embodiments of the disclosed subject matter. As opposed to the side-deployment of the device carrier 101 as shown in FIG. 2B, the alternative location 403 of the device carrier 101 may be suitable with a particular patient or user of the disclosed subject matter. In one exemplary embodiment, the device carrier 101 may be deployed in a manner similar to the deployment mechanisms used with spray jets on a bidet.

FIG. 4C shows a retracted position of the sample-collection device 100 of FIG. 1, in accordance with various embodiments of the disclosed subject matter.

As disclosed briefly above, FIG. 4D shows an alternative position of a camera 405 used to view the sample-collection device 100 of FIG. 1, in accordance with various embodiments of the disclosed subject matter. In this embodiment, the camera 405 is mounted distal to and separate from the device carrier 101. The camera 405 may be mounted, for example, to an underside of the toilet ring 211 or at another position such that the device carrier 101 is viewable by the camera 405.

FIGS. 5A and 5B show an example of a method to detect various medicines and their metabolites from a urine stream of a patient. At operation 501, a bathroom motion sensor is tripped. The motion sensor may be any type of, for example, infrared or ultrasonic motions sensor known in relevant arts that sense when a person has entered a room. For example, a motion sensor used to turn on a light switch to illuminate a room upon entry by a person may be used. In embodiments, the bathroom motion sensor may be localized to the toilet, similar to or the same as the urination-detection device 401 of FIG. 4. In embodiments, the bathroom motion sensor may be a remote control device (not shown but understandable to a skilled artisan upon reading and understanding the disclosed subject matter). The remote control may be activated by the patient when proximate to the bathroom (e.g., a button on a remote control pressed by the patient upon or shortly after entry to the bathroom). The remote control may also store biometric and/or other data pertinent to the patient such as age, sex, prescribed medications, amounts of the medications, etc.

In addition to or instead of the remote control device, the patient may enter biometric identification at operation 503. The biometric identification may be entered in one of a number of different ways. For example, the biometric identification may be entered into the system including the sample-collection device by a fingerprint scanner 303 or a retinal of facial detection scanner into an identification device 331 as described above with reference to FIGS. 3A and 3B. Therefore, at operation 501 indicating that the bathroom motion sensor is tripped may be considered optional where the patient may enter biometric identification at operation 503.

Once the biometric identification information is detected by the system including the sample-collection device, the method will be able to make a determination whether the patient is biologically male or biologically female at operation 505 and operation 511, respectively. Based on a determination that the patient is biologically male, a further determination is made whether a seat pressure sensor has been activated at operation 507. A pressure sensor (not shown but understood by a skilled artisan) may be placed onto or embedded into the toilet seat ring (e.g., the toilet ring 211 of FIG. 2B). In addition to a pressure sensor, a number of other types of sensors may be used to make a determination whether the patient is in a seated position. For example, a temperature detector (e.g., a resistance-temperature detector (RTD) or thermocouple) may be used in addition to or instead of a pressure sensor placed on or embedded into the toilet ring 211. The temperature detector will sense an increase in temperature over ambient temperature when the patient is in a seated position.

If the seat pressure sensor (or other sensor type) has not been activated at operation 507, then a motorized arm is activated (e.g., the driving component 201 of FIG. 2A) is activated and the sample-collection device 100 (see FIG. 1) is moved to a position to collect a urine sample from a standing biologically-male patient at operation 509.

At operation 515, the sample-collection device 100 may be adjusted to a final position if needed by, for example, a micro-movement adjuster. However, in various embodiments, the sample-collection device 100 may not need to be adjusted to a final position if the biologically-male patient has been instructed properly to urinate onto the absorbent pad 109 (see FIG. 1).

If at operation 511, if a determination is made that the patient is a biological female, then a motorized arm is activated (e.g., the driving component 201 of FIG. 2A) is activated and the sample-collection device 100 (see FIG. 1) is moved to a position to collect a urine sample from a seated biologically-female patient at operation 513. At operation 515, the sample-collection device 100 may be adjusted to a final position if needed by, for example, a micro-movement adjuster. In various embodiments, the sample-collection device 100 may be adjusted to collect a higher volume of urine based on, for example, a mass sensor (thereby sensing a mass flow rate (mass per unit time) of the urine stream) or acceleration sensor (thereby sensing a high or highest velocity of the urine stream) placed within or below the absorbent pad 109.

With reference now to FIG. 5B, the method continues at operation 517 where user positions and other information (e.g., such as user identification of the patient) are saved to a local storage device or remote storage device. At operation 519, a light (e.g., a green light) will be displayed to the patient to indicate that the system is ready for use. A determination is made at operation 521 to determine when urination is initiated by the patient. Once the urination process is complete (e.g., as sensed by the mass sensor or acceleration sensor described above), a signal is sent at operation 523 to operation 525.

At operation 525, a total volume of the urine sample collected is assessed and the medication adherence test is completed at operation 527. A determination as to whether the medication-compliance testing was successful i.e., performed at operation 529. In one embodiment, a determination as to whether the medication-compliance testing was successful may be, for example, that one of the expected medications or metabolites was identified or, alternatively, that no medication or metabolite was identified. A further determination may be made at optional operation 531 as to whether an adequate volume of urine was collected for a successful test. An optional step, at operation 533, alerts the user of the completion of the test and whether a recommended calibration of positioning of the testing arm (e.g., a placement of the sample-collection device 100) is recommended for a subsequent urination event.

Based on the detailed description provided herein, a person of ordinary skill in the art will recognize other combinations of the elements to produce a sample-collection device with deployment mechanisms, as well as various means to identify a user of the system and detect a flow of a urine stream. Further upon reading and understanding the disclosed subject matter, the person of ordinary skill in the art will recognize various operations and methods for using the described sample-collection device with deployment mechanisms that are within the scope of the present disclosure.

Further, although various embodiments are discussed separately, these separate embodiments are not intended to be considered as independent techniques or designs. As indicated above, each of the various portions may be inter-related and each may be used separately or in combination with other embodiments of the disclosed subject matter discussed herein. For example, although various embodiments of operations, systems, and processes have been described, these methods, operations, systems, and processes may be used either separately or in various combinations.

Consequently, many modifications and variations can be made, as will be apparent to a person of ordinary skill in the art upon reading and understanding the disclosure provided herein. Functionally equivalent methods and devices within the scope of the disclosure, in addition to those enumerated herein, will be apparent to the skilled artisan from the foregoing descriptions. Portions and features of some embodiments may be included in, or substituted for, those of others. Such modifications and variations are intended to fall within a scope of the appended claims. Therefore, the present disclosure is to be limited only by the terms of the appended claims, along with the full scope of equivalents to which such claims are entitled. It is also to be understood that the terminology used herein is for the purpose of describing particular embodiments only and is not intended to be limiting.

As used herein, the term “or” may be construed in an inclusive or exclusive sense. Further, other embodiments will be understood by a person of ordinary skill in the art based upon reading and understanding the disclosure provided. Moreover, the person of ordinary skill in the art will readily understand that various combinations of the techniques and examples provided herein may all be applied in various combinations.

Throughout this specification, plural instances may implement components, operations, or structures described as a single instance. Although individual operations are illustrated and described as separate operations, one or more of the individual operations may be performed concurrently, and, unless otherwise stated, nothing requires that the operations necessarily be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter described herein.

The Abstract of the Disclosure is provided to allow the reader to ascertain quickly the nature of the technical disclosure. The abstract is submitted with the understanding that it will not be used to interpret or limit the claims. In addition, in the foregoing Detailed Description, it may be seen that various features may be grouped together in a single embodiment for the purpose of streamlining the disclosure. This method of disclosure is not to be interpreted as limiting the claims. Thus, the following claims are hereby incorporated into the Detailed Description, with each claim standing on its own as a separate embodiment.

### The Following Numbered Examples Include Embodiments of the Disclosed Subject Matter

Example 1: In an embodiment, the disclosed subject matter includes an apparatus that can be attached to a toilet to detect various medicines and their metabolites from a urine stream of a patient. The apparatus includes a device carrier having at least one reagent-infused strip, an absorbent pad, and a transfer tube to direct at least portions of the urine stream from the absorbent pad to the at least one reagent-infused strip. The apparatus further includes a camera to collect an image from each of the at least one reagent-infused strips and produce information therefrom, and a signaling device comprising a combination transponder and signaler to transmit information received from the camera to a storage device for later analysis of the various medicines and their metabolites.

Example 2: The apparatus of Example 1, wherein the information received from the camera is arranged to be transmitted wirelessly to a storage device.

Example 3: The apparatus of any one of the preceding Examples, wherein the camera comprises a color camera arranged to collect one or more color changes on each of the at least one reagent-infused strips.

Example 4: The apparatus of any one of the preceding Examples, further including a bracket to mount the device carrier to a toilet ring, and a driving component configured to adjust the device carrier from a stored position to a position that is at least partially into the urine stream.

Example 5: The apparatus of any one of the preceding Examples, further including a biometric-identification device to identify a user of the apparatus, the biometric-identification device comprising a fingerprint scanner.

Example 6: The apparatus of any one of the preceding Examples, further including a biometric-identification device to identify a user of the apparatus, the biometric-identification device comprising at least one device selected from a facial scanner and a retinal scanner.

Example 7: The apparatus of any one of the preceding Examples, further comprising a urination-detection device, the urination-detection device to detect the presence of the urine stream of the patient.

Example 8: The apparatus of any one of the preceding Examples, wherein the camera is mounted to an underside of a toilet ring mounted on the toilet.

Example 9: In an embodiment, a method for detecting various medicines and their metabolites from a urine stream of a patient is disclosed. The method including obtaining a biometric identification of the patient, and making a determination as to whether the patient is biologically male or biologically female. Based on a determination the patient is biologically male, making a further determination whether the biologically-male patient will be urinating from a seated position or a standing position. The method further includes moving a device carrier having at least one reagent-infused strip, an absorbent pad, and a transfer tube to a proper position to capture at least a portion of the urine stream based on the determination between a seated patient and a standing patient. A determination is made as to whether a urination process is completed. A camera is used to collect an image from each of the at least one reagent-infused strips and producing information therefrom.

Example 10: The method of Example 9, further including transmitting information received from the camera to a storage device for later analysis of the various medicines and their metabolites.

Example 11: The method of either one of Examples 9 and 10, wherein the determination is made that the patient is biologically male and is seated is based on determining whether a pressure sensor is activated in a toilet seat upon which the patient is seated.

Example 12: The method of any one of Example 9 through Example 11, further including activating a micro-movement adjuster to adjust a position of the at least one reagent-infused strip, the absorbent pad, and the transfer tube to a position to capture a larger portion of the urine stream.

Example 13: The method of any one of Example 9 through Example 12, further including assessing a total volume of the urine stream that has been collected by at least one of the at least one reagent-infused strip, the absorbent pad, and the transfer tube.

Example 14: The method of any one of Example 9 through Example 13, further including making a determination as to whether a medication-adherence test was completed.

Example 15: In an embodiment, the disclosed subject matter includes a sample-collection device to detect various medicines and their metabolites from a urine stream of a patient. The device includes a device carrier having at least one reagent-infused strip to collect at least a portion of the urine stream, and a camera to collect an image from each of the at least one reagent-infused strips and produce information therefrom.

Example 16: The sample-collection device Example 15, further including an absorbent pad and a transfer tube hydraulically coupled to the at least one reagent-infused strip to direct the at least portions of the urine stream from the absorbent pad to the at least one reagent-infused strip.

Example 17: The sample-collection device of either one of Example 15 and Example 16, further including a signaling device comprising a combination transponder and signaler to transmit the information received from the camera to a storage device for later analysis of the various medicines and their metabolites.

Example 18: The sample-collection device of any one of Example 15 through Example 17, wherein the camera comprises a color camera configured to collect one or more color changes on each of the at least one reagent-infused strips.

Example 19: The sample-collection device of any one of Example 15 through Example 18, further including a bracket to mount the device carrier to a toilet ring used by the patient, and a driving component configured to adjust the device carrier from a stored position to a position at least partially into the urine stream.

Example 20: The sample-collection device of any one of Example 15 through Example 19, further including a biometric-identification device to identify a user of the device, the biometric-identification device comprising a device selected from a fingerprint scanner, a facial scanner, and a retinal scanner.

What is claimed is:

1. An apparatus that can be attached to a toilet to detect various medicines and their metabolites from a urine stream of a patient, the apparatus comprising:
a device carrier having at least one reagent-infused strip, an absorbent pad, and a transfer tube to direct at least portions of the urine stream from the absorbent pad to the at least one reagent-infused strip;
a camera to collect an image from each of the at least one reagent-infused strips and produce information therefrom; and
a signaling device comprising a combination transponder and signaler to transmit information received from the camera to a storage device for later analysis of the various medicines and their metabolites.

2. The apparatus of claim 1, wherein the information received from the camera is configured to be transmitted wirelessly to a storage device.

3. The apparatus of claim 1, wherein the camera comprises a color camera configured to collect one or more color changes on each of the at least one reagent-infused strips.

4. The apparatus of claim 1, further comprising:
a bracket to mount the device carrier to a toilet ring; and
a driving component configured to adjust the device carrier from a stored position to a position that is at least partially into the urine stream.

5. The apparatus of claim 1, further comprising a biometric-identification device to identify a user of the apparatus, the biometric-identification device comprising a fingerprint scanner.

6. The apparatus of claim 1, further comprising a biometric-identification device to identify a user of the apparatus, the biometric-identification device comprising at least one device selected from a facial scanner and a retinal scanner.

7. The apparatus of claim 1, further comprising a urination-detection device, the urination-detection device to detect the presence of the urine stream of the patient.

8. The apparatus of claim 1, wherein the camera is mounted to an underside of a toilet ring mounted on the toilet.

9. A method for detecting various medicines and their metabolites from a urine stream of a patient, the method comprising:
obtaining a biometric identification of the patient;
making a determination as to whether the patient is biologically male or biologically female;
based on a determination the patient is biologically male, making a further determination whether the biologically-male patient will be urinating from a seated position or a standing position;
moving a device carrier having at least one reagent-infused strip, an absorbent pad, and a transfer tube to a proper position to capture at least a portion of the urine stream based on the determination between a seated patient and a standing patient;
determining that a urination process is completed; and
using a camera to collect an image from each of the at least one reagent-infused strips and producing information therefrom.

10. The method of claim 9, further comprising transmitting information received from the camera to a storage device for later analysis of the various medicines and their metabolites.

11. The method of claim 9, wherein the determination is made that the patient is biologically male and is seated is based on determining whether a pressure sensor is activated in a toilet seat upon which the patient is seated.

12. The method of claim 9, further comprising activating a micro-movement adjuster to adjust a position of the at least one reagent-infused strip, the absorbent pad, and the transfer tube to a position to capture a larger portion of the urine stream.

13. The method of claim 9, further comprising assessing a total volume of the urine stream that has been collected by at least one of the at least one reagent-infused strip, the absorbent pad, and the transfer tube.

14. The method of claim 9, further comprising making a determination as to whether a medication-adherence test was completed.

15. A sample-collection device to detect various medicines and their metabolites from a urine stream of a patient, the device comprising:
a device carrier having at least one reagent-infused strip to collect at least a portion of the urine stream; and
a camera to collect an image from each of the at least one reagent-infused strips and produce information therefrom.

16. The sample-collection device of claim 15, further comprising an absorbent pad and a transfer tube hydraulically coupled to the at least one reagent-infused strip to direct the at least portions of the urine stream from the absorbent pad to the at least one reagent-infused strip.

17. The sample-collection device of claim 15, further comprising a signaling device comprising a combination transponder and signaler to transmit the information received from the camera to a storage device for later analysis of the various medicines and their metabolites.

18. The sample-collection device of claim 15, wherein the camera comprises a color camera configured to collect one or more color changes on each of the at least one reagent-infused strips.

19. The sample-collection device of claim 15, further comprising:
a bracket to mount the device carrier to a toilet ring used by the patient; and
a driving component configured to adjust the device carrier from a stored position to a position at least partially into the urine stream.

20. The sample-collection device of claim 15, further comprising a biometric-identification device to identify a user of the device, the biometric-identification device comprising a device selected from a fingerprint scanner, a facial scanner, and a retinal scanner.
